(19) United States
(12) Patent Application Publication
(10) Pub. No.: US 2009/0255026 A1
(43) Pub. Date: Oct. 15, 2009

(54) EYE SHADE HAVING CONFORMABLE NOSE PIECE

(76) Inventor: Devin Benner, Pleasant Ridge, MI (US)

Correspondence Address: BUTZEL LONG;IP DOCKETING DEPT, 350 SOUTH MAIN STREET, SUITE 300, ANN ARBOR, MI, 48104, US

(21) Appl. No.: 12/102,044

(22) Filed: Apr. 14, 2008

(51) Int. Cl. A61F 9/00

(52) U.S. Cl. 2/12

(57) ABSTRACT

An eye shade is provided having a first side, a second side, a first eye portion and a second eye portion form from the first side and the second side, a nose portion formed from the first side and the second side, and a conformable nose piece associated with the nose portion wherein the conformable nose piece is adapted to allow the nose portion of the eye shade to be conformed to the user's nose to substantially prevent light from passing between a user's face and the eye shade.

### BACKGROUND OF THE INVENTION

The invention generally relates to eye shades, and more particularly, to an eye shade designed to prevent light leakage reaching the users eyes by providing a customizable seal between the user's nose and the eye shade.

Eye shades are commonly used to assist people obtaining restful sleep by preventing the transmission of light to the user's eyes. Many different eye shade designs have been utilized in attempts to minimize the transmission of light to the user's eyes, including the eye shade disclosed in U.S. Pat. No. 6,745,397, hereby incorporated by reference. However, one problem that continues to plague these eye shade devices, is that the eye shade is unable to completely conform to the topography of the user's face, especially about the nose region, and thus allows light to reach the user's eyes thereby reducing the effectiveness of the eye shade in providing restful sleep.

Previous eye shade designs have attempted to prevent this light leakage by adding additional padding to the mask in the nose region of the eye shade and/or by recessing the mask to fit around the user's nose. While this additional padding was able to prevent at least some of the passage of light in that region, the variability of the sizes of the faces of the vast number of potential users made it difficult to fabricate a universal eye shade. Also, the additional padding imparts discomfort to the user of the eye shade, preventing the user from obtaining restful sleep while wearing the eye shade.

Accordingly, it would be advantageous and desirable to have an eye shade design which prevents light from entering at the nose region of the eye shade by providing a customizable fit between the user's nose and the eye shade while being comfortable for the user to sleep in.

### SUMMARY OF THE INVENTION

An eye shade is provided having a first side, a second side, a first eye portion and a second eye portion form from the first side and the second side, a nose portion formed from the first side and the second side, and a conformable nose piece associated with the nose portion wherein the conformable nose piece is adapted to allow the nose portion of the eye shade to be conformed to the user's nose to substantially prevent light from passing between a user's face and the eye shade.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates a front view of an embodiment of the invention having outer convex eye portions extending away from the front portion.

FIG. 1A illustrates a cross-sectional view of the embodiment illustrated in FIG. 1.

FIG. 1B illustrates a cross-sectional view of an alternative embodiment of the invention.

FIG. 1C illustrates a side view of an eye portion of the embodiment illustrated in FIG. 1.

FIG. 2 illustrates a rear view of the embodiment illustrated in FIG. 1.

FIG. 3 illustrates an alternative embodiment of the invention.

FIG. 3A illustrates a cross-sectional view of the embodiment illustrated in FIG. 3.

### DETAILED DESCRIPTION OF THE INVENTION

The invention generally relates to an eye shade that can be conformed or customized to fit the user's nose and thereby prevent light from entering the eye shade. Referring to the figures, exemplary embodiments of the invention will now be described. The exemplary embodiments are provided to illustrate the invention and should not be construed as limiting the scope of the invention.

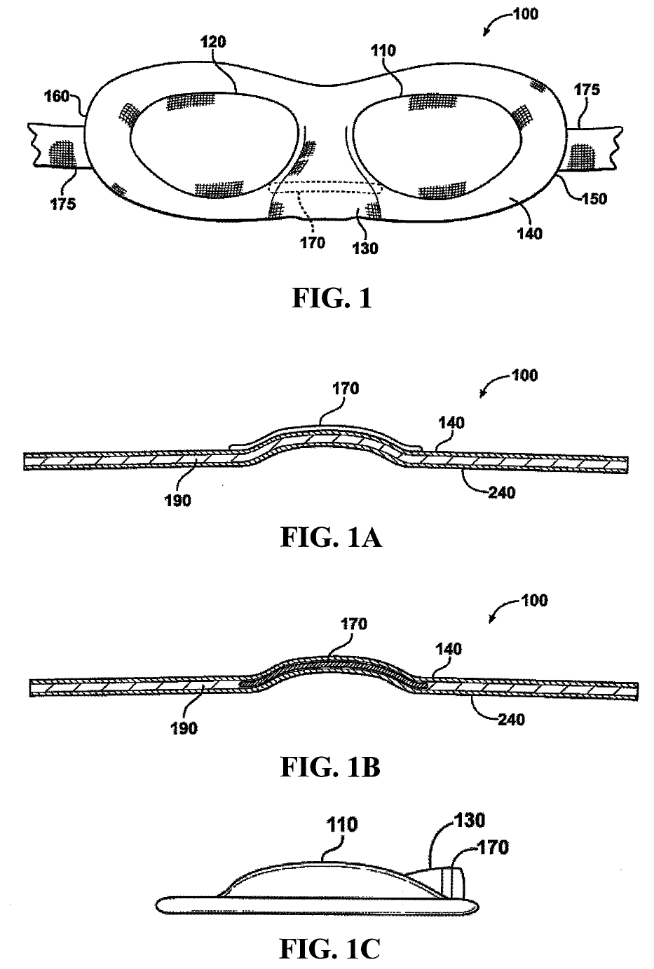

FIG. 1 illustrates a front view of an embodiment of the invention. In this embodiment of the invention, eye shade 100 comprises left outer eye portion 110, right outer eye portion 120, outer nose portion 130 and front portion 140. Left outer eye portion 110 and right outer eye portion 120 are convex in shape extending away from front portion 140. Outer nose portion 130 is convex in shape extending away from front portion 140.

A conformable nose piece 170 is associated with the nose portion 130. The conformable nose piece 170 is adapted to allow the nose portion 130 of the eye shade 100 to be conformed to a user's nose to substantially prevent light from passing between a user's face and the eye shade 100.

The nose piece 170 can be formed of any suitable moldable material which is capable of retaining its conformation when it is bent or molded into a new form. Suitable materials for the conformable nose piece 170 include metal strips, such as those made of aluminum, other suitable metals, or suitable polymeric materials. Additionally, other materials having “a memory” which would allow them to be bent or conformed about the nose of the user can also be utilized.

Referring to FIG. 1A, the conformable nose piece 170 can be disposed on the front portion 140 of the mask 100 and can be affixed by any suitable method including the use of a suitable adhesive, stitching or sewing, and/or bonding. As shown in FIG. 1A, the conformable nose piece 170 can be disposed in between the front portion 140 and the rear portion 240 in an intermediate or inner portion 190. The conformable nose piece 170 can be affixed within the inner portion by any suitable means including those discussed immediately above for affixation to the front portion 140. The nose piece 170 can also be disposed on the rear portion 240.

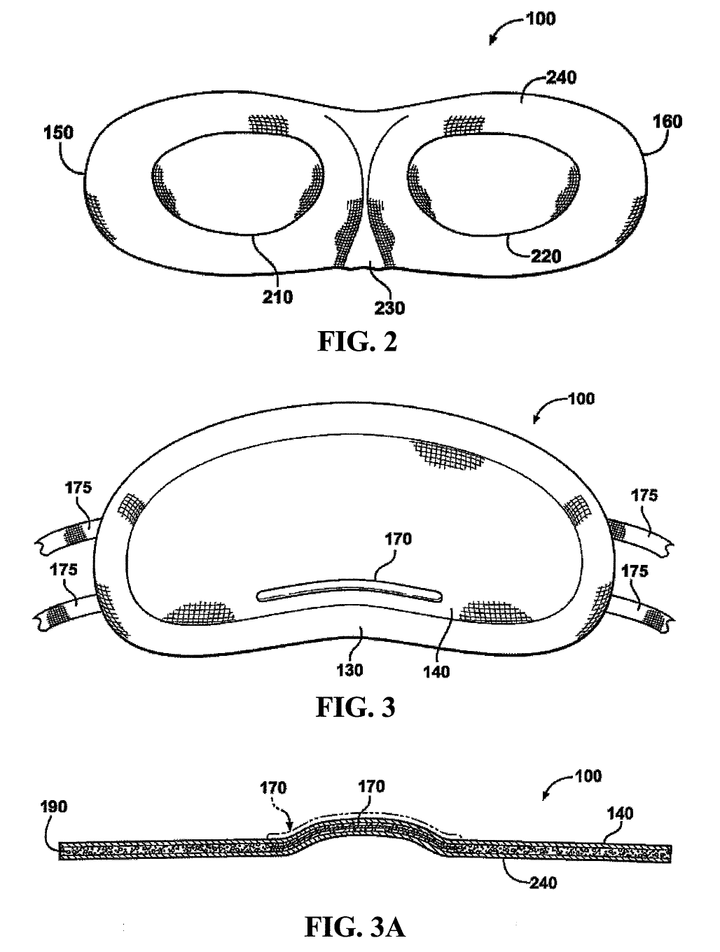

FIG. 2 illustrates the rear view of eye shade 100. Left inner eye portion 210 is the inner compliment to left outer eye portion 110. Right inner eye portion 220 is the compliment to right outer portion 120. Left inner eye portion 210 and right inner eye portion 220 are both concave shaped. Rear portion 240 is the compliment to front portion 140. Inner nose portion 230 is the compliment to outer nose portion 130. Inner nose portion 230 is concave in shape.

Left inner eye portion 210 and right inner eye portion 220 can vary in length, width and depth dimensions to prevent various sized eyelashes and eye portions of users from coming into contact with the surface of either left inner eye portion 210 or right inner eye portion 220. FIG. 1C illustrates a side view of an eye portion of the embodiment illustrated in FIG. 1. Only one eye portion is illustrated as the left and right eye portions are symmetrical. The depth h (illustrated in FIG. 1C) of either left inner eye portion 210 or right inner eye portion 220 can be a depth in the range of three-fourths (¾) of an inch to three and a half (3½) inches at the center of either inner left eye portion 210 or inner right eye portion 220. That is, different embodiments can have different depth h to accommodate various length eye lashes or enhanced eyelashes (such as false or fake eyelashes). Eye shade 100 can vary in dimension so as to accommodate various facial shapes and sizes.

Eye shade 100 can have both front portion 140 and rear portion 240 comprised of suitable man-made material that can be formed over an inner portion lying between front portion 140 and rear portion 240 that is comprised of suitable compressible material, such as a foam rubber type material. The composition of front portion 140, rear portion 240, and inner portion 190 (illustrated in FIG. 1A) lying between front portion 140 and rear portion 240 are such that eye shade 100 can be formed by a heat source in a press which molds the composite materials. Since eye shade 100 is comprised of molded material, eye shade 100 has “memory”. Therefore, eye shade 100 can be folded, twisted, washed, etc., and will retain its formed shape.

Eye shade 100 can have varying means 175 for positioning on or attaching to a face. Such means include a single strap, a plurality of coupled straps, etc. These positioning/attaching means can be made of a single elastic piece attached to right end side 160 and left end side 150, or separate pieces wherein complimentary pieces are each coupled to right end 160 and left end 150. For embodiments of eye shade 100 having a plurality of positioning/attaching pieces, various means can be used for fastening each of the plurality of pieces together. These means include, VELCRO® hook and loop fasteners, metal connectors, plastic connectors, buckles, snaps, etc. The positioning pieces can be adjustable and also vary in length and width, to accommodate various sized users.

In this embodiment the nose portion, comprising outer nose portion 130 and inner nose portion 230, can be varied in size to accommodate various sized noses. In another embodiment of the invention, the nose portion can be partially or entirely removed.

FIG. 3 illustrates an alternative embodiment of the present invention. In this embodiment, an eye shade 100 is shown which possesses a traditional shape eye shade. This embodiment does not necessarily include the contours incorporated into the embodiment shown in FIGS. 1-2. It is to be understood that in this alternative embodiment, like numerals are used to identify portions of this embodiment which are common between this embodiment and the embodiment set forth in FIGS. 1-2.

Referring to FIG. 3A, as with the embodiment shown in FIGS. 1-2, a conformable nose piece 170 can be disposed on either the front portion 140 or the mask 100 (as shown in phantom) or disposed in contact with the intermediate or inner layer 190 as shown.

The invention has been described in an illustrative manner, and it is to be understood that the terminology which has been used is intended to be in the nature of words of description rather than of limitation.

Obviously, many modification and variations of the present invention are possible in light of the above teaching. It is, therefore, to be understood that within the scope of the appended claims, the invention may be practiced otherwise than as specifically described.

What is claimed:

1. An eye shade comprising:
a first side;
a second side;
a first eye portion and a second eye portion formed from the first side and the second side;
a nose portion formed from the first side and the second side; and
a conformable nose piece associated with the nose portion wherein the conformable nose piece is adapted to allow the nose portion of the eye shade to be conformed to a user's nose to substantially prevent light from passing between a user's face and the eye shade.

2. An eye shade according to claim 1 further including an intermediate layer coupled to the first side and the second side.

3. An eye shade according to claim 1, wherein the conformable nose piece is formed of a moldable material.

4. An eye shade according to claim 3, wherein the moldable material is a metal strip.

5. An eye shade according to claim 1, wherein the eye shade further includes a strap adapted to retain the eye shade on the user.

6. An eye shade according to claim 2 further comprising a first outer-eye portion and a second outer eye portion formed from the first side, the intermediate layer and the second side, and the first and second outer eye portions extend outward, a first inner eye portion and a second inner eye portion formed form the first side, the intermediate layer and the second side, the first and second inner eye portions do not come in contact to a user's surrounding eye area to avoid comprising eye makeup, eye makeup including eyebrow, eye liner and eye shadow makeup, wherein the first outer eye portion and the second outer eye portion are convex shaped, said first side and said second side are each made from the same type of material, and light cannot pass through said eye shade.

7. An eye shade according to claim 6, wherein the first inner eye portion and the second inner eye portion are concave shaped.

8. An eye shade according to claim 7, wherein the first inner eye portion and the second inner eye portion have a depth such that a person wearing the eye shade avoids contact with the first and second inner eye portions.

9. An eye shade according to claim 1, wherein the conformable nose piece is disposed on the first side.

10. An eye shade according to claim 1, wherein the conformable nose piece is disposed on the second side.

11. An eye shade according to claim 2, wherein the conformable nose piece is disposed in contact with the intermediate layer.
